(19) United States
(12) Patent Application Publication
(10) Pub. No.: US 2005/0234377 A1
(43) Pub. Date: Oct. 20, 2005

(54) Vacuum orthosis and associated methods

(75) Inventors: Augusto Sarmiento, Miami, FL (US); William S. Patterson, Orlando, FL (US)

Correspondence Address: William S. Patterson;Suite 305, 1809 E. Broadway St., Oviedo, FL, 32765, US

(73) Assignee: VACUITY, INC., Orlando, FL (US)

(21) Appl. No.: 11/020,739

(22) Filed: Dec. 23, 2004

Related U.S. Application Data
(60) Provisional application No. 60/532,255, filed on Dec. 23, 2003.

(51) Int. Cl. A61F 5/00

(52) U.S. Cl. 602/19

(57) ABSTRACT

The vacuum orthotic device includes the use of an inner sleeve, a single or multi-piece shell, and an outer sleeve. The shell is positioned between the sleeves and typically includes a fabric liner or other porous material to act as a wick. A valve is provided in the outer sleeve for drawing air from between the inner and outer sleeves, i.e. to create a vacuum. The sleeves overlap and sealingly contact each other at the proximal and distal ends of the orthotic device. A disconnectable hand-pump or otherwise actuated pump may be used to connect to the valve and draw the vacuum between the sleeves. The device effectively prevents the loss of firm contact between the treatment site and the device.

### CROSS REFERENCE TO RELATED APPLICATIONS

This application claims the benefit of U.S. Provisional Application No. 60/532,255 filed on Dec. 23, 2003.

### FIELD OF THE INVENTION

The present invention relates to the field of orthotics, and, more particularly, to an orthotic device including a vacuum feature to effectively prevent the loss of firm contact between the treatment site and the device.

### BACKGROUND OF THE INVENTION

Bracing is a technique used with great frequency in the management of a large variety of medical conditions, that is, traumatic, developmental, degenerative and congenital. Braces are used as stabilizing or corrective devices in patients of all ages. Braces are also used to lessen pain and disability as in the case of the painful back, or for the prevention and/or correction of progressive deformities, as in the case of scoliosis. Similarly, they are used for the prevention and control of progression in the case of the osteoporotic spine or the spine affected with neoplasms of various types.

Another area where braces are widely used is in the treatment of fractures. They are used either as initial stabilizers or as devices to prevent deformity at the fracture site, while permitting the functional use of the injured extremity or area.

For example, U.S. Pat. No. 6,066,107 to Habermeyer discloses a double-walled cuff-forming cushion which can be deformed into a sleeve. A plurality of loose particulate filling bodies is provided in the inner space of the cuff-forming cushion between the two walls and the cushion can be consolidated by evacuation into a stiff structure. An outer plastic shell is secured to the sleeve with hook and loop fasteners.

Typically, when using an orthosis it is important to maintain close contact between the treatment area and the orthotic device. Otherwise, the inevitable reduction in the degree of swelling that necessarily follows an injury, produces a loss of “fit” of the brace, resulting in the creation of deformities at the fracture site. With traditional braces, frequent tightening of the fit of the appliance is strongly urged. However, compliance with such a request is frequently ignored, or the value of the advice is not truly understood.

Accordingly, there is still a need for an orthotic device to effectively prevent the loss of firm contact between the treatment site and the device.

### SUMMARY OF THE INVENTION

The present invention includes a vacuum system and orthotic device used in the treatment of fractures to effectively prevent the loss of firm contact between the injured limb and the brace, therefore minimizing and/or eliminating the traditional adverse changes.

The invention applies to orthotic devices used in the treatment of other conditions such as spine pain or fractures, post-operative conditions such as spine fusion or laminectomies. The invention equally applies to braces used after joint surgery or various types, such as arthroscopy, internal fixation of fractures of the upper and lower extremities, foot and hand.

The present invention is directed to a vacuum orthotic device and method including the use of an inner sleeve, a single or multi-piece shell, and an outer sleeve. The shell is positioned between the sleeves and typically includes a fabric liner or other porous material to act as a wick. A valve is preferably provided in the outer sleeve for drawing air from between the inner and outer sleeves, i.e. to create a vacuum. The sleeves overlap and sealingly contact each other at the proximal and distal ends of the orthotic device.

The sleeves are preferably made of polyurethane, and may be tubular and slidable onto the patient's arm or leg, or the sleeves may be wrap-around type sleeves with a sealing edge, e.g. sealing tape, to ease the installation of the sleeves and brace. The shell may be a multi-piece unit including anterior and posterior shell portions which are fit to provide some overlap with each other at the edges. Alternatively, the shell may be a single piece unit with an opening, and flexible enough to allow opening of the shell for installation on the patient's arm or leg, for example. Such a single piece unit may also include an integrated hinge, such as a thinned wall portion opposite the opening to ease the opening of the shell.

A disconnectable hand-pump or otherwise actuated pump may be used to connect to the valve and draw the vacuum between the sleeves. Furthermore, an external, internal or integrated tank or rigid reservoir may be included to hold a vacuum.

A method aspect of the present invention includes treating a body portion with the orthotic device. The method includes surrounding the body portion with an inner sleeve, positioning an outer sleeve over the inner sleeve and sealingly contacting the inner sleeve at proximal and distal ends thereof and defining a vacuum chamber therebetween. A hard shell is positioned between the inner and outer sleeves and includes a porous shell liner adjacent the inner sleeve. The vacuum chamber is evacuated via a valve in the outer sleeve.

### BRIEF DESCRIPTION OF THE DRAWINGS

FIG. 1 is a schematic diagram illustrating a tubular inner sealing sleeve applied to a patient's leg in accordance with the present invention.

FIG. 2 is a schematic diagram illustrating a two-piece shell being applied over the inner sealing sleeve of FIG. 1.

FIG. 3 is a schematic diagram illustrating a two-piece shell applied over the inner sealing sleeve of FIG. 1 and including an overlap area.

FIG. 4 is a schematic diagram illustrating an outer sleeve applied over the shell and inner sleeve, and including a valve in accordance with the present invention.

FIG. 5 is a schematic diagram illustrating a wrap-around type inner and/or outer sleeve in accordance with the present invention.

FIG. 6 is a schematic diagram illustrating a wrap-around type inner sleeve applied to a patient's leg in accordance with the present invention.

FIG. 7 is a schematic diagram illustrating a single-piece shell prior to being applied to a patient's leg in accordance with the present invention.

FIG. 8 is a schematic diagram illustrating a single-piece shell applied over the inner sealing sleeve on a patient's leg in accordance with the present invention.

FIG. 9 is a schematic diagram illustrating the use of a pump in connection with the orthotic device applied to a patient's leg in accordance with the present invention.

FIG. 10 is a schematic diagram illustrating the use of a pump and an external vacuum tank in connection with the orthotic device in accordance with the present invention.

FIG. 11 is a schematic diagram illustrating the use of an internal or integrated vacuum reservoir in connection with the orthotic device in accordance with the present invention.

### DETAILED DESCRIPTION

The present invention will now be described more fully below with reference to the accompanying drawings, in which preferred embodiments of the invention are shown. This invention may include different embodiments and should not be construed as limited to the embodiments set forth below. These embodiments are provided so that the disclosure will be thorough and complete, and will fully convey the scope of the invention to those skilled in the art. Like numbers refer to like elements throughout the description.

The present invention includes a vacuum system and orthotic device or brace used in the treatment of a body portion, such as a fractured extremity, to effectively prevent the loss of firm contact between the body portion and the device, therefore minimizing, if not eliminating the traditional adverse changes resulting in a loss of fit, for example. The following embodiment is described with respect to a fractured leg; however, the invention applies to various body portions including the trunk, neck and extremities.

The invention applies to orthotic devices used in the treatment of other conditions such as spine pain or fractures, post-operative conditions such as spine fusion or laminectomies. The invention equally applies to braces used after joint surgery or various types, such as arthroscopy, internal fixation of fractures of the upper and lower extremities, foot and hand.

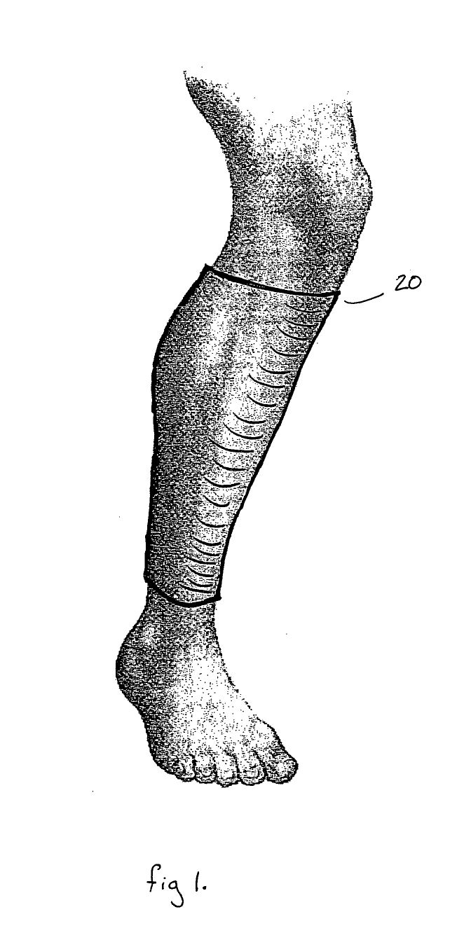

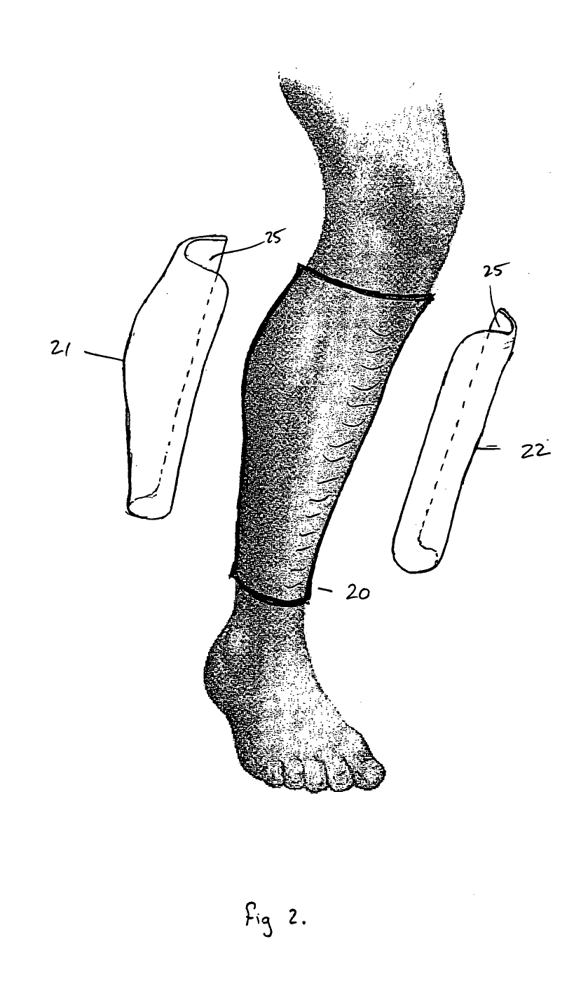

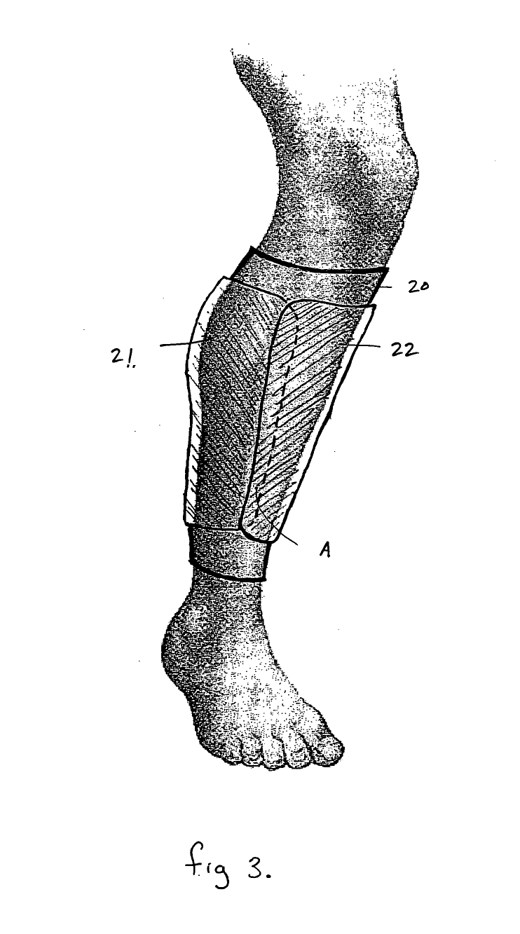

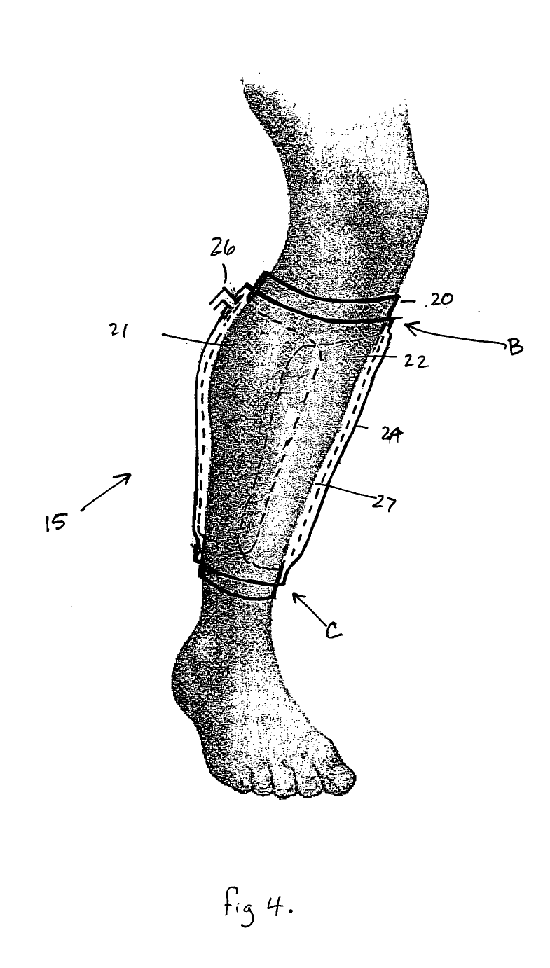

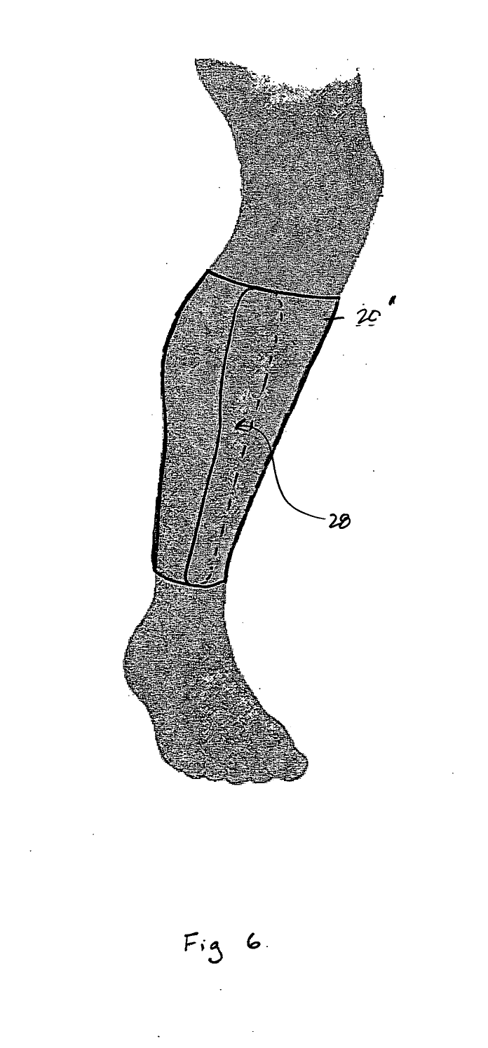

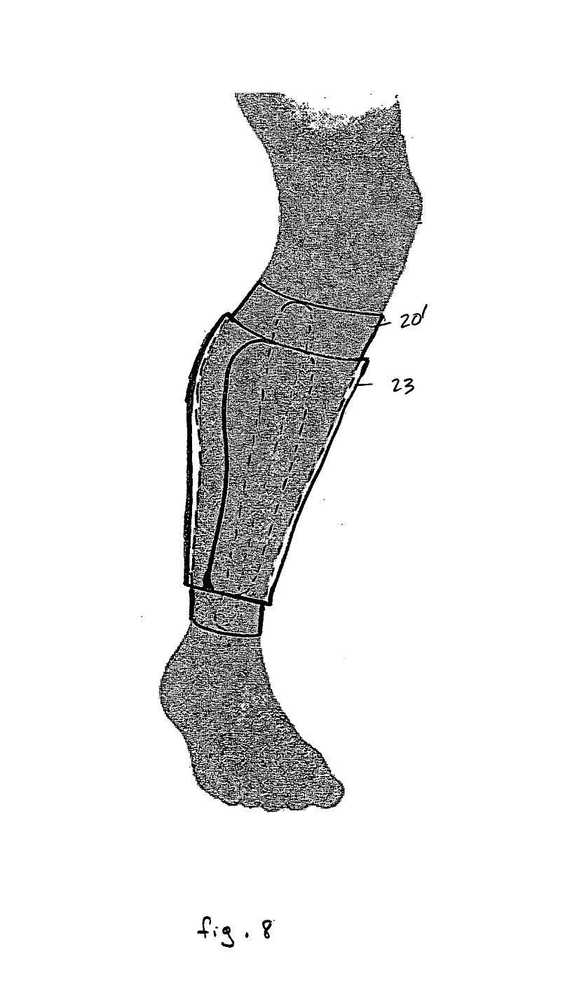

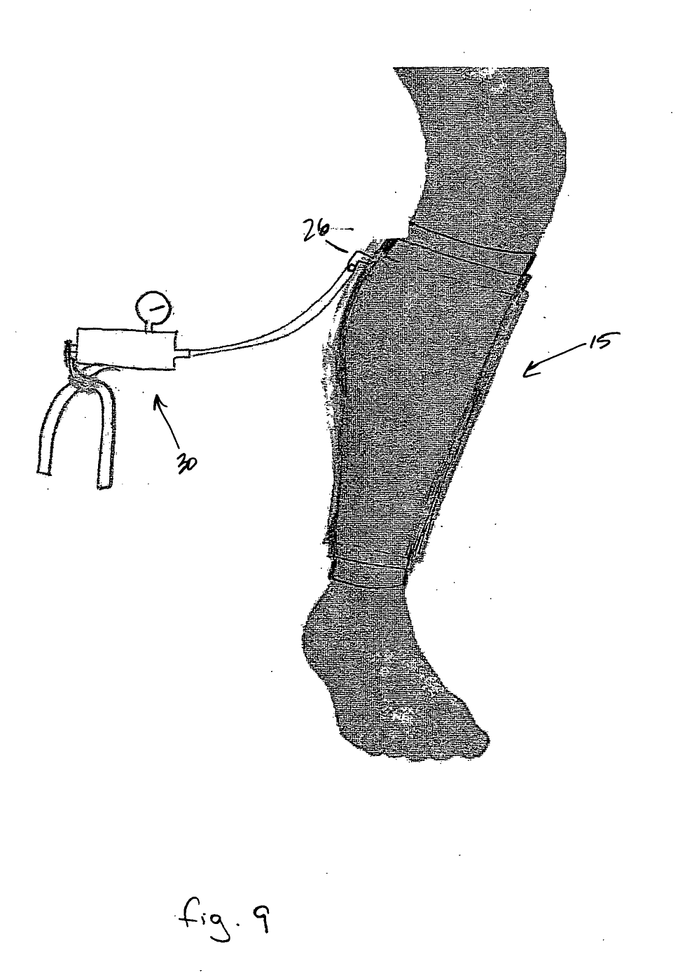

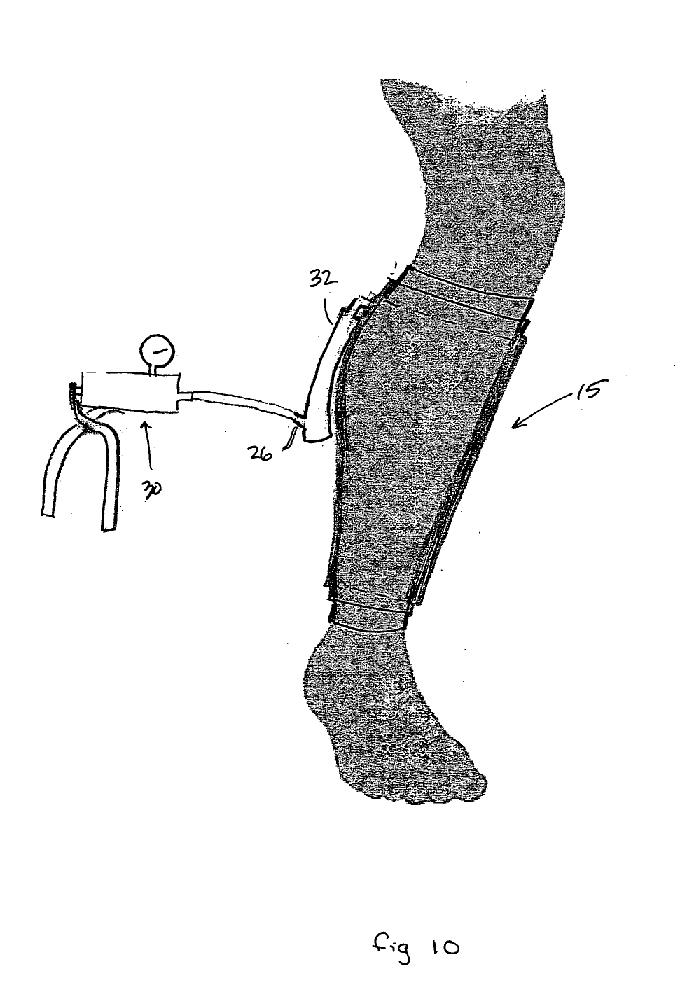

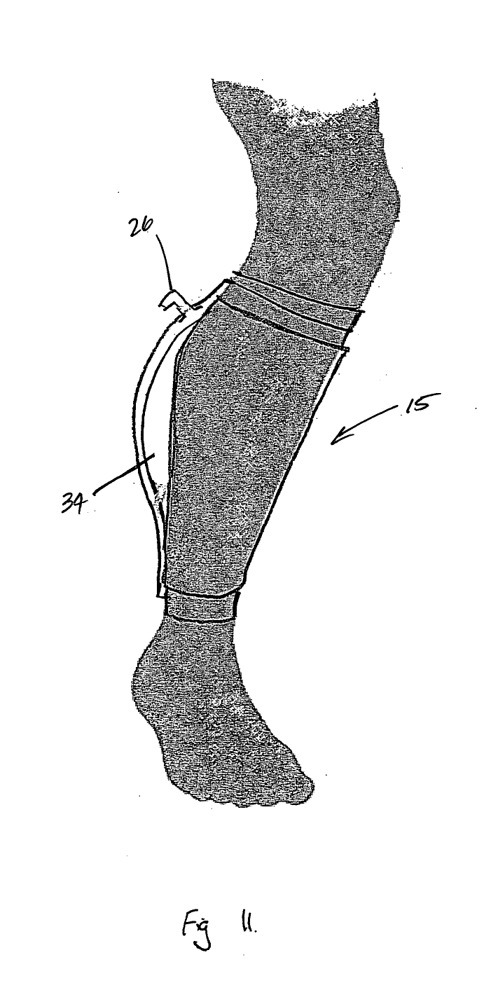

Referring now to FIGS. 1-11, a vacuum orthotic device and method in accordance with the present invention will now be described. The orthotic device 15 includes the use of an inner sleeve 20, a single 23 or multi-piece 21/22 shell, and an outer sleeve 24. The shell is positioned between the sleeves and typically includes a fabric liner 25 or other porous material to act as a wick. A valve 26 is preferably provided in the outer sleeve 24 for drawing air from between the inner and outer sleeves, i.e. to create a vacuum. The sleeves overlap and sealingly contact each other at the proximal and distal ends of the orthotic device and thereby define a vacuum chamber 27 therebetween.

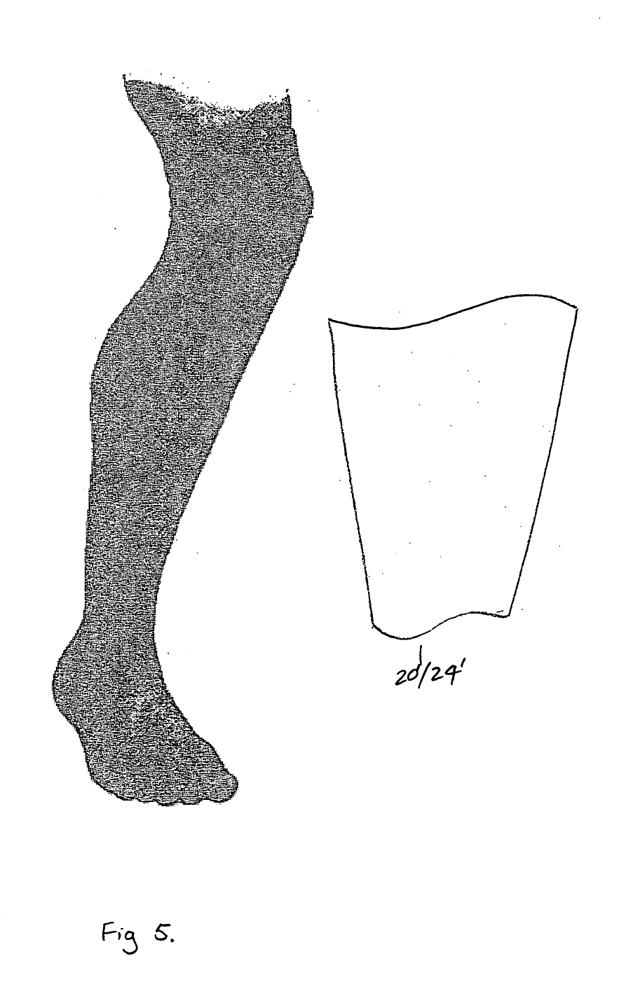

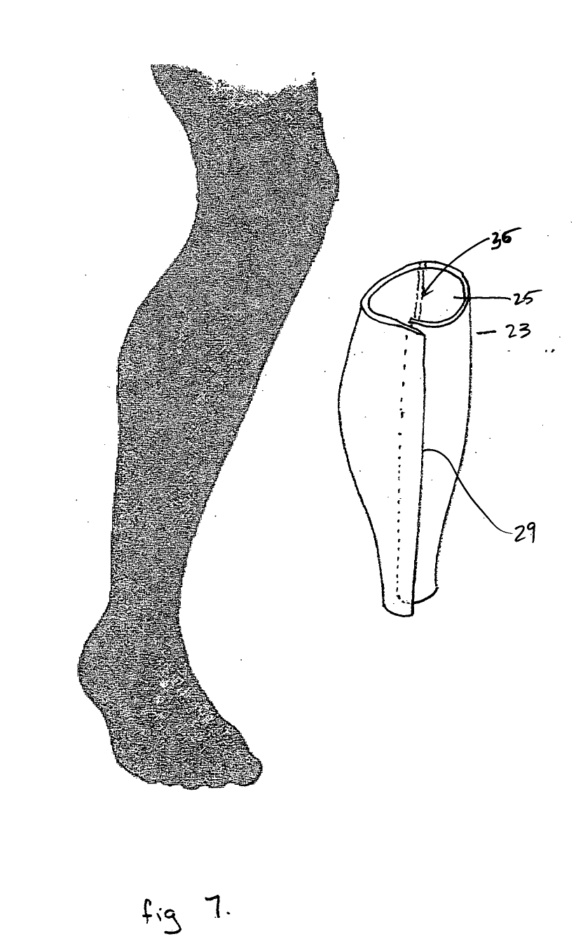

The sleeves are preferably made of polyurethane, and may be tubular and slidable onto the patient's arm or leg, or the sleeves may be wrap-around type sleeves 20′/24′ (e.g. as shown in FIGS. 5, 6 and 8). with a sealing edge 28, e.g. sealing tape, to ease the installation of the sleeves and brace. The shell may be a multi-piece unit including anterior 22 and posterior 21 shell portions which are fit to provide some overlap A with each other at the edges. Alternatively, the shell may be a single piece unit (e.g. as shown in FIG. 7) with an opening 29, and flexible enough to allow opening of the shell for installation on the patient's arm or leg, for example. Such a single piece unit may also include an integrated hinge 36, such as a thinned wall portion opposite the opening to ease the opening of the shell.

A disconnectable hand-pump 30 or otherwise actuated pump may be used to connect to the valve 26 and draw the vacuum between the sleeves. Furthermore, an external 32, internal 34 or integrated tank or rigid reservoir may be included to hold a vacuum.

A method aspect of the present invention includes treating a body portion with the orthotic device 15. The method includes surrounding the body portion with an inner sleeve 20, positioning an outer sleeve 24 over the inner sleeve and sealingly contacting the inner sleeve at proximal B and distal C ends thereof and defining a vacuum chamber 27 therebetween. A hard shell 21/22 or 23 is positioned between the inner and outer sleeves and includes a porous shell liner 25 adjacent the inner sleeve. The vacuum chamber 27 is evacuated via a valve 26 in the outer sleeve.

Advantages of the orthotic device of the present invention include, but are not limited to: volume control; wound healing; maintaining intimate fit of brace and improving patient compliance with fracture orthosis due to the importance of total contact and an intimate fit with the fracture orthosis over the effected site. In the past as the patient's affected area would lose contact due to volume loss and the squeezing effect of the brace at the site, the patient would have to tighten the brace up by hand which resulted in patient compliance issues and only a certain amount of tightening could be accomplished by the patient or practitioner.

With the orthotic device of the present invention, i.e. the Sarmiento Vacuum Orthosis, the tissue rehydration is being accomplished by the vacuum effect being placed on the effected site keeping loss of fluid to that area to a minimum. This in turn improves healing and provides another option to surgery.

The above presents a description of the best mode contemplated for a vacuum orthotic device according to the present invention, and of the manner and process of assembling and using it, in such full, clear, concise, and exact terms as to enable any person skilled in the art to which it pertains to make and use this device. The embodiments described herein are, however, susceptible to modifications and alternate constructions which are fully equivalent. Consequently, it is not the intention to limit this invention to the particular embodiments disclosed. On the contrary, the intention is to cover all modifications and alternate constructions coming within the spirit and scope of the invention as generally expressed by the following claims, which particularly point out and distinctly claim the subject matter of the present invention.

1. An orthotic device for treatment of a body portion comprising:
an inner sleeve to surround the body portion;
an outer sleeve positioned over the inner sleeve and sealingly contacting the inner sleeve at proximal and distal ends thereof and defining a vacuum chamber therebetween;
a hard shell positioned between the inner and outer sleeves and comprising a porous shell liner adjacent the inner sleeve; and
a valve in the outer sleeve for evacuating the vacuum chamber.

2. The orthotic device according to claim 1 wherein the valve comprises a one-way valve.

3. The orthotic device according to claim 1 wherein the inner and outer sleeves comprise polyurethane sleeves.

4. The orthotic device according to claim 1 wherein the inner and outer sleeves comprise tubular sleeves slidable onto the body portion.

5. The orthotic device according to claim 1 wherein the inner and outer sleeves comprise wrap-around sleeves for wrapping onto the body portion.

6. The orthotic device according to claim 5 wherein the inner and outer sleeves further comprise an overlapping sealing edge.

7. The orthotic device according to claim 6 wherein the sealing edge comprises sealing tape.

8. The orthotic device according to claim 1 wherein the hard shell comprises a multi-piece shell including respective overlapping portions.

9. The orthotic device according to claim 8 wherein the multi-piece shell comprises an anterior shell portion and a posterior shell portion.

10. The orthotic device according to claim 1 wherein the hard shell comprises a single-piece shell having an opening and a flexibility to allow the shell to be installed onto the inner sleeve on the body portion.

11. The orthotic device according to claim 10 wherein the single piece shell comprises an integrated hinge.

12. The orthotic device according to claim 1 further comprising a rigid vacuum reservoir connected between the valve and the vacuum chamber.

13. A method of treating a body portion with an orthotic device comprising:
surrounding the body portion with an inner sleeve;
positioning an outer sleeve over the inner sleeve and sealingly contacting the inner sleeve at proximal and distal ends thereof and defining a vacuum chamber therebetween;
positioning a hard shell between the inner and outer sleeves and comprising a porous shell liner adjacent the inner sleeve; and
evacuating the vacuum chamber via a valve in the outer sleeve.

14. The method according to claim 13 wherein the inner and outer sleeves comprise polyurethane sleeves.

15. The method according to claim 13 wherein the inner and outer sleeves comprise tubular sleeves slidable onto the body portion.

16. The method according to claim 13 wherein the inner and outer sleeves comprise wrap-around sleeves for wrapping onto the body portion.

17. The method according to claim 16 wherein the inner and outer sleeves further comprise an overlapping sealing edge.

18. The method according to claim 13 wherein the hard shell comprises a multi-piece shell including an anterior shell portion and a posterior shell portion having respective overlapping portions.

19. The method according to claim 13 wherein the hard shell comprises a single-piece shell having an opening and a flexibility to allow the shell to be installed onto the inner sleeve on the body portion.

20. The method according to claim 19 wherein the single piece shell comprises an integrated hinge.

21. The method according to claim 13 further comprising a rigid vacuum reservoir connected between the valve and the vacuum chamber.
